(19) United States
(12) Patent Application Publication
(10) Pub. No.: US 2008/0314286 A1
(43) Pub. Date: Dec. 25, 2008

(54) Reactive Particles, Production Thereof, and Use Thereof in Kits and Cements

(75) Inventors: Carsten Gellermann, Gerbrunn (DE); Herbert Wolter, Tauberbischofsheim (DE); Thomas Ballweg, Wertheim (DE)

Correspondence Address: GUDRUN E. HUCKETT DRAUDT, SCHUBERTSTR. 15A, WUPPERTAL, 42289, DE

(73) Assignee: Fraunhofer-Gesellschaft zur Forderung der angewand e.V., Munchen (DE)

(21) Appl. No.: 12/091,750

(22) PCT Filed: Nov. 7, 2006
(86) PCT No.: PCT/EP2006/068186

§ 371 Date: Apr. 28, 2008

(30) Foreign Application Priority Data
Nov. 10, 2005 (DE) 10 2005 053 721.9

(51) Int. Cl. A61K 6/06

(52) U.S. Cl. 106/35

(57) ABSTRACT

Ionomer particles with an inner area and an outer area are provided. The outer area has an oxidic matrix with cations selected by observing the following conditions. Only two different types of cations must be present that belong to one of the following groups (a) to (d), and these two types of cations must be selected from two different ones of the groups (a) to (d): (a) ions of the elements of the second main group and bivalent ions of the transition elements and the lanthanides; (b) ions of the elements of the third main group with the exception of boron and trivalent ions of the transition elements and the lanthanides; (c) ions of the elements of the fourth main group with exception of carbon and tetravalent ions of the transition elements and the lanthanides (d) ions of the elements of the fifth main group, selected from ions of antimony and bismuth and pentavalent ions of the transition elements. Particles of the composition ZrO₂—Y₂O₃, ZrO₂—Al₂O₃, BaToO₃, Al₆Si₂O₁₃ are not contained.

The present invention concerns inorganic or optionally organically modified particles (ionomer particles) that can be subjected in a targeted way to leaching of certain cations and that are therefore suitable for use as an inorganic component in so-called glass ionomer cements. The invention further relates to a method for producing these particles as well as their use in ionomer cements.

The term “ionomer particle” is to be understood in the aforementioned context as inorganic particles that in combination with a preferably acid-containing matrix can be used in multiple ways as cements (self-curing, light-curing etc.). In order for a cement-forming reaction to take place at all, these particles must be instable in a defined or targeted way, i.e., in combination with water in the presence of the partner with which they are to be reacted they must release metal ions that lead to a curing reaction in the partner substance.

Classic glass ionomer cements with purely inorganic curing action, light-curing glass ionomer cements (with additional organic polymerizable components) and so-called compomers (the term is derived by contraction of the expressions composite and ionomer and is meant to refer to such cements in which the carboxyl group is bonded to the same molecule that also carries a cross-linkable double bond; see e.g. “Glass ionomers, The Next Generation”, Proc. of the 2nd Int. Symp. on Glass Ionomers, 1994, page 13ff) are often used as filler material in particular in dentistry. As a partner for curing (cement formation) the aforementioned poly alkene acids are usually used. Advantages of these materials are: no or hardly any shrinkage up to an expansion caused by the ionomer reaction as a result of absorbing water; unproblematic incorporation of fluorides and phosphates; excellent bonding to the tooth tissue (or also to other body tissues such as bone) as a result of the acid groups in the matrix, simple application. The basic structure of the usually glass-like ionomers is comprised of a ternary system of silicon dioxide/aluminum oxide/calcium oxide. By melting together these components, particles are obtained which in the presence of, for example, poly alkene acids undergo a two-stage reaction. By means of the attack of protons of the poly alkene acids, calcium ions are first leached from the glass composite and, in an instable phase or so-called primary curing, they are complexed by the carboxylate groups of the poly alkene acids. The secondary curing then leads to a stable phase in which also aluminum cations migrate out of the glass ionomer. Aluminum polyalkenoates are formed as a result of hydration of the poly salts.

Disadvantages of the glass ionomer cements are the strength that is still too minimal, the high wear, and the insufficient x-ray absorption that are all caused by the currently employed classic ground glass ionomer particles.

The known ionomer particles can be obtained inter alia by co-melting the respective starting compounds (mainly oxides). The grinding process to which the melted glass ionomer is subjected in order to obtain the desired particles however promotes the generation of sharp-edged non-round particles. In this way, the resulting wear resistance of the ionomer state is unsatisfactory. The formed particles are heterodisperse and relatively large and must therefore generally be subjected to a complex classification process in order to obtain them in at least somewhat acceptable size distribution. In addition to a high labor expenditure this means also a high material loss and thus extremely bad yield. The aluminum silicate matrix is often not homogeneous as a result of co-melting. For example, embedding of fluorides occurs in the form of calcium fluoride-rich droplets.

Other cements for dental purposes that are obtained by grinding, melting and/or sintering, have also disadvantages. WO 00/071082 discloses the use of a composition of the formula 3 CaO.SiO₂ that is known in the construction industry as a ceramic material in medicine, wherein the formula must be strictly observed. This material is also known as Portland cement. By observing specific grain sizes and grain size distributions that can be obtained however only by a complex method with several grinding and firing processes to be performed in an inert gas atmosphere (argon) as well as intermediate quenching steps and by using very high temperatures (up to approximately 1,250° C.), this material according to the aforementioned document is well suited for specific medical purposes.

A significant improvement relative to the particles that are ground and/or fired from glasses or other materials are glass ionomer particles obtained by way of wet-chemical routes (for example, sol-gel technologies). For example, WO 00/05182 discloses particles obtained in this way that have a spherical or approximately spherical shape. These ionomer particles are either purely inorganic particles but they can also be organically modified.

The ionomer particles that are producible according to the aforementioned WO 00/05182 contain, like “classic” ionomer particles, at least three cationic components in their outer area, i.e., silicon ions, ions that can occupy the lattice sites of the silicon by generating a negative charge excess, for example, aluminum, and ions selected from those of the elements of the first and second main groups as well as other elements that can be present in bivalent form and can compensate the negative charge excess. e.g. calcium. It has been found in this context that in the ionomer reaction in addition to primary and secondary curing as described above also the outer shell of the ionomer particles can be dissolved by proton attack with formation of ortho silicic acid. This ortho silicic acid condenses in the further course of reaction to silica gel; a gel layer is formed.

Further developments in the field of ionomer cements are desirable primarily because making available dually curable glass ionomer cements in the long run can lead to a boom for the already existing applications in the medical field as, for example, dental filling material, bone cement, and adhesives. For this purpose, inexpensive ionomer particles that can be produced in a simple way are required.

The inventors of the present invention have now surprisingly found that even those particles that do not contain more than two different cation types that are hardly soluble as salts of poly alkene acids can be used as ionomer particles in the glass ionomer cement reaction when these particles are produced by wet chemistry (for example the sol-gel route), As a result of the wet-chemical preparation, the particles have a larger and more reactive surface area in comparison to conventionally produced particles (produced by firing and/or grinding from glasses or other oxides). Because they must not be melted, in some circumstances they may provide also improved interlinking with the polymer matrix. In particular, the inventors have found that a network based on SiO₂ is not required in the inorganic matrix of particles into which, as disclosed in the prior art, the other components are embedded. The presence of Si—O portions can even reduce reactivity which is not always desirable.

Accordingly, the invention provides ionomer particles with an inner area and an outer area that are characterized in that the outer area comprises an oxidic matrix with cations whose selection must observe the following conditions:
two different types of cations must be present;
the two types of cations must be selected from two different ones of the following groups (a) to (e):
(a) ions of elements of the second main group as well as bivalent ions of the transition elements and the lanthanides,
(b) ions of the elements of the third main group with the exception of boron and optionally aluminum as well as trivalent ions of the transition elements and of the lanthanides,
(c) ions of the elements of the fourth main group with exception of carbon and optionally of silicon as well as tetravalent ions of the transition elements and the lanthanides,
(d) ions of the elements of the fifth main group selected from ions of antimony and bismuth,
from a third of the afore mentioned groups no further cations may be present in the oxidic matrix.

The oxidic matrix is preferably homogeneous or substantially homogeneous. Excluded from the range of protection for the particles themselves are however those particles whose oxidic matrix is comprised of calcium oxide and silicon oxide in a molar ratio of 3:1 or contain this mixture, at least when the particles are produced starting from calcium carbonate and finely dispersed silicon dioxide (silica gel) by a method that requires the application of temperatures above 1,000° C. These particles are disclosed in WO 00/71082. Excluded are preferably moreover particles of a combination of aluminum oxide or silicon dioxide with an oxide selected from oxides of lanthanum, zirconium and yttrium, optionally also zinc, tantalum, tin, ytterbium, barium, and strontium. Some of the aforementioned particles are disclosed in U.S. patent application 2002/002214 A1 as filler material for dental materials. However, these materials are not designed to be a component of an ionomer cement. Accordingly, in this document any suggestion is lacking that such particles could be instable in the presence of suitable matrices.

The invention provides the possibility of making available or employing ionomer particles in a substantially easier way than before and with elimination of at least one component, and thus less expensively, and with great variety. As a result of the wet-chemical production, for example, by sol-gel route, the energy costs for the production are also minimal because no high melting or sintering temperatures must be employed.

The ionomer particles according to the invention can be particles with a (completely or substantially) homogeneous matrix of a mixed oxide of the two aforementioned ion types that optionally contain particulate inclusions (for example, fluoride salt(s); phosphate) and/or are surface-modified. In this case, the inner and the outer areas of the particles are identical. Alternatively, the particles can be of the core-shell type. In this case, the matrix surrounds as mentioned above a core that deviates with regard to its composition from that of the matrix. The composition of the core is not critical because this part of the particles does not participate in the ionomer reaction. It can therefore be selected, for example, for the purpose of imparting to the particles additional properties such as x-ray opacity or the like. Of course, these particles can also have particulate inclusions in their matrix area or can be modified on their exterior as described above for the homogeneous particles.

In a preferred embodiment the ionomer particles contain also fluoride ions. Fluoride ions promote tooth health by a remineralization effect (apatite formation).

In another also preferred embodiment of the invention the ionomer particles contain moreover phosphate ions. In addition to their availability for the purpose of remineralization (biocompatibility), by means of these ions the cement reactivity can also be affected in an advantageous way in that, in addition to the standard additives, they provide a further instrument for adjusting the time periods important for the application, such as processing time and curing time, and that also have a positive effect in regard to adhesion to tooth and bone.

In three independent preferred embodiments of the invention the oxidic matrix of the particles is formed of a combination of elements of the groups (a) and (b), the groups (a) and (c), as tell as the groups (b) and (c). Among these combinations that of the groups (a) and (b) is especially preferred. Even more preferred are particles that contain calcium ions.

The individual inventive particles have preferably a spherical or approximately spherical shape. Particle mixtures should preferably have a narrow particle distribution. Their size is usually, but not necessarily, within a nanometer to micrometer range. The particle size can be adjusted to e.g. between 5 nm and 50 μm, It is preferred to provide relatively small particles because they have more surface area. In this way, the reactivity is increased and thus hardening of the cement is accelerated or improved. A further advantage of smaller particles is an improved translucence of the resulting cement. Examples of particle sizes are, for example, 20 nm to 20 μm or 0.5 μm to 50 μm. The respectively selected particle size can be realized in this context within a narrow distribution sector that is significantly below an order of magnitude. Smaller particles, for example, in the range of 50 nm to 1 or 2 μm are especially suitable as a tooth filling material. In addition to the already described advantages, for small particles also a particularly high proportion of ionomer can be incorporated. In order to provide an especially high proportion of ionomer in the cement, in a special embodiment of the invention a mixture of two or three batches of ionomer particles are provided that, with regard to their defined narrow size distribution, respectively, have such a size ratio relative to one another that the smaller particles can fit in the gaps of an imaginary dense sphere packing of the larger particles and the optionally present much smaller particles fit in gaps of the resulting packing. This configuration is also particularly suitable for tooth fillings because a high ionomer proportion in the cement can be realized and mechanically demanding dental cements should contain particle contents as high as possible. The bulk density is a parameter that provides information in regard to the packing behavior of particles. In this way, it is possible to determine early on in which way high particle contents can be obtained. However, in addition to relatively small particles, according to the invention also larger particles should be made available, be it as the largest batch of a mixture of sizes, as described above, be it for utilization of the cements in other medical or non-medical fields (for example, as bone substance or an adhesive).

In a preferred embodiment the particles are functionalized on the surface which promotes the prevention of agglomerate formation.

In another preferred embodiment the particles are porous. Porous particles (i.e. particles with pores on the outer surface) have as a result of the higher number of atoms on the outer surface of the particles a higher ion release/leaching in the presence of water and thus an improved glass ionomer reaction. Porous particles however also have disadvantages: they produce a mechanically less stable cement. Therefore, the degree of porosity is selected in accordance with the desired purpose. Moreover, a reduced porosity can be selected when the particles as a result of the ions used for this purpose are especially reactive, i.e., can be leached especially well in the ionomer cement.

The porous glass ionomer particles have preferably a pore volume of 0.001 to 2.0 cm³/g, preferably 0.01 to 1.5 cm³/g, and especially preferred 0.1 to 1.0 cm³/g. The pores are produced by wet chemistry in low-temperature processes (sol-gel technology emulsion processes etc.). Under the wet-chemical reaction conditions clusters or primary particles of the size 1 to 10 nm are formed first that during the further course of reaction build a network (gel). Depending on conditions of after treatment, the porous gel network can be densified or compacted more or less. For a temperature range of below 500° C. greatly porous systems result while at temperatures above 800° C. in general almost non-porous but still generally amorphous glass ionomer particles are produced. In the temperature range above 1,000° C. particle systems are obtained that have a glass-like to ceramic character. By appropriate selection of the temperature for after treatment the porosity can be adjusted accordingly in the desired way wherein, in particular, when the temperature is set within a range between 500° C. to 800° C. transitional forms between greatly porous and less porous particles are obtained.

By aerosol methods at high temperatures particles can be produced that have preferably a minimal to small porosity. At temperatures between 1,000 to 1.600° C. the formation of sinter necks is observed first followed by “merging” of the individual components. In these high-temperature processes the pores will disappear almost completely and dense particles are produced primarily.

The particles can contain additives in homogeneous form or as particulate inclusions in the aforementioned matrix. These additives can be for example provided for increasing the x-ray absorption capability or a change of color, transparency or reflective index.

Both particle variants, the homogeneous mixed particles as well as the particles of the core-shell type can be produced by means of the sol-gel technology or other wet-chemical routes such as emulsion, aerosol, inkjet or Stöber methods. In this context, the particles of the core-shell type can be produced more elegantly and less expensively because it is possible in their case to apply on an optionally very inexpensive core (e.g. of SiO₂) a shell of the mixed oxidic matrix that is relatively variable with regard to thickness. The thickness of the shell, depending on the COOH contents of the matrix, can be adjusted. Preferably, it is at least 1-10 nm, even more preferred at least 10-50 nm so that in cross-section radially outwardly at least approximately 50, preferably at least approximately 100 metal atoms M of the existing two types are present on the core. i.e., the thickness of the shell is thus at least 50-100 times the radius of an M-O group. An upper limit must not be observed because, as mentioned above, only the outer atom layers determine the chemistry of the ionomer particles. Additives as the above described ones can be present in the core and/or in the shell. This provides the possibility of combining two additives that are possibly not really compatible with one another in one type of particles.

The production of the spherical ionomer particles is realized as mentioned above particularly by way of the different wet-chemical methods. In this context, a dispersion containing an organic component is formed in which a controlled hydrolysis and condensation occur. The expression “dispersion” is used in this context even though possibly also true solutions, suspensions or emulsions can be obtained or produced in certain states of the hydrolytic condensation. Also, sol and gel formation processes are to be included in this expression (for example; the disperse phase of a dispersion or emulsions can gel). This term therefore is to be understood to have a relatively broad meaning. The dispersion can be transformed in different ways, for example, by the so-called Stöber process or spray drying into spherical particles. As an organic component at least one compound is employed that is selected from organic compounds of the cations of the elements listed under (a) to (d). The expression “organic compound” is to be understood as any “organometallic” compound that comprises at least one organic component bonded by oxygen to the metal or a complexed organic component or an organic component bonded to the metal such that in the presence of water, aqueous or other solvents or dispersion agents (e.g. alcohols) at least a partial hydrolysis of this compound will be initiated which optionally will be started only under the effect of acid or base, whereupon the compound is subjected to a controlled condensation so that chain condensates or cross-linked condensates are formed in the “solvent” but no uncontrolled precipitation reaction occurs (the expression “solvent” is to be understood of course such that the agent in general will not provide a true solution of the organic compound or compounds; usually a suspension, a dispersion, an emulsion, a sol or a gel is formed). Examples of organic compounds are e.g. oxo complexes such as alcoholates or carboxylates but also other suitable metal complexes or organometallic compounds. As needed, both cations of the future spherical particles can be used in the form of so-called organic compounds.

When a cation of the elements mentioned under (a) is used as an organic component, not only, but particularly, the carboxylates and alcoholates are suitable. Especially preferred are magnesium, calcium, and strontium acetate and the alcoholates, for example, isopropanolate, of these elements. Further examples are calcium acetyl acetonate or calcium oxalate. When instead this cation is not to be employed as an organic compound, the use in the form of optionally extremely fine powders of the corresponding inorganic compounds, for example, the oxides, halogenides (chlorides, fluorides), phosphates or other salts (e.g. Ca(NO₃)₂, MgCl₂, SnCl₂) that are soluble or insoluble in the selected solvents, is suitable. Because these powders possibly will not completely dissolve and therefore clusters, for example, oxide clusters, with primarily one cation type may remain, the oxidic matrix possibly cannot be entirely homogeneous. It is therefore also referred to as “substantially homogeneous” The clusters should advantageously have a diameter of less than 50 nm; in general they will be smaller than 10 nm.

The metals among which the cations of the group mentioned under (a) can be selected, comprise e.g. beryllium, magnesium, calcium, strontium, and barium but also strontium, tin or zinc (the latter in their bivialent form). By selecting the suitable cations, specific properties can be generated in a targeted way, for example, x-ray opacity, reactivity, optical properties or the like.

When a cation of the elements mentioned under (b) is to be used as an organic component, preferably oxo complexes are used for this purpose. As an oxo complex; e.g. alcoholates, diketonates; and carboxylates are suitable. As examples of alcoholates ethanolate, secondary and tertiary butylates, for example, of aluminum, should be mentioned. Examples of carboxylates are those of oxalic acid or methacrylic acid. Acetates or acetyl acetonates as well as further complexes with chelate forming agents are also suitable. When instead this cation is not to be used in the form of an organic component, the use in the form of optionally extremely fine powders of the corresponding inorganic compounds, for example, the oxides, halogenides (chlorides, fluorides), phosphates or other salts (e.g. AlCl₃) that are soluble or insoluble in the selected solvents, is suitable. Further examples are ethyl aluminum dichloride, iron(III)fluoride, iron(III)citrate, iron acetyl acetonate.

The elements that can be used under (b) are preferably those of the third main group, including gallium, indium and thallium. Also, trivalent niobium, trivalent tantalum, scandium; yttrium, and rare earth elements such as lanthanum; cerium, gadolinium, ytterbium are suitable. By selecting special elements, for example, very heavy elements, certain properties such as x-ray opacity can be produced. Aluminum is suitable in this connection only to a limited extent. Depending on the selection of the second component and the degree of porosity and thus of the reactivity of aluminum containing particles; their ion release rate can be so high that not in all cases a drop below a satisfactory safety spacing relative to the toxicity limit is ensured.

Examples of starting compounds for incorporation of cations of the group (c) are titanium(IV) butoxide, zirconium butoxide; zirconium acetate, n-butyl tin trichloride; tin(IV) acetate, tin(IV) sulfate.

As already mentioned above; the use of silicon as element of the group (c) is less beneficial because possibly a reactivity reduction must be contended with. However if for certain considerations silicon is still to be used as an element of the group (c); for example, in combination with another especially reactive partner, and this cation is to be used in the form of an organic component, there are different possibilities to incorporate the silicon ions into the ionomer particles. For example, hydrolyzable silanes or siloxanes can be added to the dispersion, for example, alkyl and/or alkoxy silanes. In this case, particles with a homogeneous silicate-containing matrix are obtained. Alternatively, to a dispersion with compounds of the complexed elements of the group (a), (b), or (d), for example, a second dispersion of silicon dioxide particles with very minimal diameter can be added. In this case, the silicon dioxide forms cluster-like structures within the outer area of the particles that are being formed that, based on the minimal diameter, are crosslinked very well with the oxide of the other element.

Examples of starting compounds for the incorporation of cations of the group (d) are tantalum(IV)butoxide, tantalum(V)chloride, ammonium heptafluoro tantalate(V).

When particles are to produced of two cation types, selected from silicon, aluminum, and calcium, despite the above-mentioned limitations, as starting compounds alkoxy silanes, aluminum alcoholates, and calcium acylate are suitable. In particular, aluminum butylate can be used with calcium acetate or aluminum butylate or calcium acetate, each in combination with silicon dioxide.

When the oxidic matrix of the ionomer particles is to contain phosphate, it can be added in the form of triethyl ortho phosphate. Fluoride incorporation can be realized by means of hexafluoro silicic acid or ammonium fluoride.

The above-mentioned dispersion, depending on the application, can have further substances admixed. An example is the incorporation of tin dioxide particles into a sol containing the aforementioned components. In this way the spherical particles with an inner area (core) of tin dioxide can be obtained that ensure e.g. excellent x-ray absorption. The core of the ionomer particles can be comprised instead also of silicon dioxide. For this purpose, silicon dioxide particles of a suitable size (for example, with a diameter of 30-100 nm (for example, for the dental field) or of 1 to 2 μm) are brought into contact with the dispersion so that the dispersion can deposit on the outer area about the core of silicon dioxide. The aforementioned ionomer-reactive modifications are only listed as examples, Many possible variants can be realized as long as the ionomer particles in their outer area have the aforementioned ionomer-reactive components.

The aforementioned organically modified components for producing the dispersion can be introduced for example into water and optionally acetic acid or glacial acetic acid can be added (or introduced into the already acidified solvent). Also, use of basic solvents is possible. Alternatively, to the organically modified components, for example, in a non-aqueous dispersion agent, e.g. alcohol, in a suitable way a quantity of water and optionally base or acid as a catalyst, which quantity is sufficient for the required hydrolysis process, can be added. Before or subsequently, the inorganic substances that optionally are also to be processed can be incorporated, which inorganic substances optionally are dissolved or dispersed prior to incorporation. In this environment a hydrolytic condensation of the organically modified components is initiated wherein however based on the reaction conditions it must be taken care that hydroxides or oxides will not precipitated in an uncontrolled fashion. Instead, transformation into chains and/or a network in which the existing van-der-Waals bonds are sufficient for obtaining a stable scaffold beyond the spatial area where the particles develop (i.e., a dispersion or suspension) or through the entire liquid (with formation of a sol or gel).

Based on the above described components a dispersion is formed that can be transformed subsequently into preferably spherical or approximately spherical particles or from which such particles can be separated. This can be realized in different ways known to a person skilled in the art. With regard to this, reference is being had to the disclosure of WO 00/05182 in which a plurality of suitable methods with literature reference are mentioned.

According to the invention it is possible, for example, by means of a method based on the Stöber process to apply onto different inert particle cores (for example, SiO₂, SnO₂ cores) a shell containing silicon ions that contains additional elements of the group (a) or the group (b) or optionally also group (d). As cores any monodisperse spherical seeds produced in any suitable way can be utilized. Inter alia, commercially available agglomerate-free, monodisperse spherical SiO₂ particles (e.g. Ludox, Fa. DuPont) or SnO₂ particles can be used. Based on the aforementioned Stöber process, also monodisperse spherical SiO₂ cores in a size range of 50 to 2,000 nm can be produced that are then provided with a “shell”.

For applying the shell, as starting materials organosilicon compounds such as alkoxy silanes in combination with compounds of the group (a) or the group (b) (the latter tvo in organic or inorganic form), optionally of the group (d) instead, can be used. The organic compounds or a low-molecular weight condensation product thereof are added, for example to 1-40% by weight of a solvent, preferably alcohol. This solution is titrated to the mother dispersion of the cores such that during the course of the growth process of the particles an oversaturation concentration that would lead to formation of new particles is not reached. Since according to this method the organic compounds are to be hydrolyzed, water is added in a concentration that is matched to the concentration of the educts. Since the hydrolysis/condensation reactions proceed under neutral conditions very slowly, an acidic or alkaline medium is preferred. A pH of 8-9 is advantageous for a uniform growth of the particles and provides monodisperse ionomer particles of an almost ideal spherical shape. They are characterized by a surprisingly fast ionomer reaction.

An in-situ surface modification is achieved inter alia by adding a silane, for example, of amino propyl triethoxy silane or methacryl oxy propyl trimethyl silane, in the form of a 1-100% by weight solution to the dispersion. As a solvent preferably the same solvent as that of the dispersion is used, for example, ethanol. Also possible is a subsequent surface modification of the dried particles. For this purpose, to the particle powder, suspended in approximately 10% by weight in an organic solvent, for example, toluene an amount of silane required for monomolecular occupation is added, optionally a catalyst is added and optionally the reaction is carried out under reflux.

Furthermore, it was found that emulsion methods are also well suited for producing the above described ionomer particles. Suitable are O/W as well as W/O methods, Preferably, the W/O method is used (see, for example, EP 0 363 927). The proportion of an aqueous phase is preferably at approximately 15 to 45 volume %, that of the emulsifying agent preferably at approximately 1 to 20% by weight. During the course of the emulsion process, a precipitation or gel formation takes place in the water droplets that preferably is triggered by a basic pH value displacement. As starting compounds salts and organic complexes of the above described elements, preferably nitrates, alcoholates, and acetates, are suitable as well as dispersions already produced therefrom without limitation. The obtained ionomer particles have surprisingly a narrow size distribution that can be significantly below an order of magnitude.

The afore described liquid can instead also be subjected to an aerosol treatment, in particular, spray drying. For example, very finely dispersed SiO₂ particles or silicon alkoxides can be mixed with alcoholates or carboxylates of the cations of the groups a) or b) in aqueous solution at pH<7. With the aid of suitable jets droplets are sprayed that have a spherical shape. They can be optionally dried, for example, at approximately 250° C. until the volatile organic compounds are removed.

All methods have in common that the obtained particles after removal of the solvent or after separation from the solvent can be subjected, if desired, to pyrolysis and sintering in as much as organic components are still present (for example, at 400° C. to 600° C.) In this way, hydrocarbon-free ionomer particles are produced. With a careful application of higher temperatures starting at approximately 500° C. into a range of approximately 800° C. the degree of porosity of the particles can be reduced in a targeted way. Temperatures above approximately 1,000° C. are often undesirable because the individual particles will melt irreversibly to an aggregate.

Depending on the employed starting compounds in the aforementioned method ionomer particles of different structure are formed. The particles can have a continuous homogeneous area of calcium silicates, strontium silicates, aluminum silicates or the like. The ionomer particles can be comprised exclusively of these structures or can have a discrete inner area that has a different composition, for example, silicon dioxide, tin dioxide, a mixture of both, aluminum silicate or the like. In a specific embodiment the spherical ionomer particles are comprised of an inner area and several outer areas that are preferably shell-like. They can be produced, for example, in that the silicon dioxide particles of a suitable size are coated with a first gel or sol, dried and optionally pyrolyzed whereupon the resulting particles are coated with a second gel or sol of a different composition, dried again and optionally pyrolyzed. At least the outermost gel or sol must have in this context a composition as described above. Even though in general this may not be required, the aforementioned spherical ionomer particles according to the invention can also be conventionally silanized or surface-modified in another way.

When the ionomers according to the invention are incorporated into matrix systems, preferably in acid-containing matrix systems, according to the above described two-step curing process cement-like materials, for example, composites, cements, compomers are produced. Their properties can be adjusted in a targeted way, as described, by utilization of corresponding starting substance, for example, by addition of x-ray-opaque components or by reaction conditions (for example, concentration, temperature, pH) with which the diameter of the particles can be varied. These materials are in particular useful in dentistry (for example, as a filing material) and in the medical sector (for example, as a bone cement). Furthermore, materials can be produced with differently adjusted transparency, color, refractive index.

The ionomer particles according to the invention can be incorporated into a variety of different organic or partially organic matrices with which they undergo a glass ionomer reaction (cement formation), Glass ionomer cements are formed by the reaction of inorganic glass ionomer particles with an acid-containing matrix system in the presence of water. The acid-containing matrix system can be of organic nature and is then generally a carboxyl-group containing polymer matrix system, for example, one of one (or several) poly alkene acid(s). The matrix system can be a homopolymer or a copolymer of unsaturated mono-, di-, or higher polycarboxylic acids (e.g., mono- di-, or tricarboxylic acids) and their anhydrides or mixtures thereof. Hydroxy carboxylic acids such as citric acid or tartaric acid can be added to the acid-containing matrix system.

As select examples polyacrylic acid, poly itaconic acid, and poly maleic acid should be mentioned. But other acids such as poly phosphonic acids of e.g. vinyl phosphonic acid, allyl phosphonic acid, vinyl benzyl phosphonic acid etc. or poly phosphonic acid esters and poly phosphoric acid esters are in principle suitable as a matrix. The matrix system can also be an acid-containing inorganic-organic hybrid polymer (eg. ORMOCER, trademark of the Fraunhofer-Geselischaft, München). Alternatively or additionally, the matrix system can also contain polymerizable monomers that can be transformed by a curing reaction (e.g. UV-induced, light-induced, redox-induced) into a polymer system. In a special embodiment, the proportion of these monomers is very high (up to 100%) so that the glass ionomer reaction is affected by the monomers and the polymerization conditions. But other preferred acidic matrix systems are also possible for exampile those with poly phosphonic acids such as poly (vinyl phosphonic acid), systems that contain additional light-curable components or matrices that can form with ionomer particles the above described compomers.

Well-suited acid-containing matrix system for the glass ionomer particles of the present invention have preferred molecular weights that, for example, for polyacrylic acid are at 200 to 200,000, particularly preferred at 5,000 to 50,000. For other poly acids, the molecular weights can be optionally calculated accordingly. For molecular weights that are too great gel formation can result that prevents the further glass ionomer reaction between the particles and the acid-containing matrix and impairs the compressive strength of the cement.

The mixture ratio (mass ratio) of acid to particles is beneficially at 0.001:1 to 10:1 and preferably between 1:5 and 5:1. When the latter ratios are surpassed or not reached, in many cases excess proportions of acid or base can be generated in the cement which, for example, with regard to desired biocompatibility or the dentin adsorption capability, can have negative effects.

As matrix systems with which the ionomer particles according to the invention are processed to cements, the acidic systems already discussed above in detail are suitable. They can be made available in aqueous phase or freeze-dried; in the latter state, water must be of course added for mixing with the ionomers.

The glass ionomer reaction is carried out with excess water as reaction medium. The mixture ratio of water to the glass ionomer particles and acid-containing matrix is preferably 0.01 to 100, especially preferred 0.1 to 10. Even though the concrete water contents in many cases can be critical, precise limits can hardly be provided because the water contents greatly depends on the composition, particle size, porosity and specific surface area of the glass ionomer particles. When the water content is too minimal, the release of ions is too low so that a satisfactory application and satisfactory mechanical properties such as compressive strength are not enabled. But water contents that are too high can also be critical because as a result of ion release that is too high the formation of loose gel networks may result. Gels, as explained above, are undesirable especially because of their bad mechanical properties.

The reaction between the particles and the acid-containing matrix is preferably carried out in a normal reactor or in a small shaking device (e.g. VOCO Mix 10), A reaction in autoclaves is also possible. Temperature from room temperature to 80° C. are suitable; preferably, temperatures of 20 to 40° C. are selected.

When the reaction times are relatively long, accelerators can be used. Preferably, these are complexing agents, for example, citric acid or tartaric acid, up to a contents of 15% by weight, preferably up to approximately 5% by weight. Other additives such as stabilizers, detergents, dispersion agents, pigments etc. are possible. Moreover, other fillers, i.e., inactive and active fillers, can be added to the reactive glass ionomer particles. This is always suitable when a porosity of the cement that is too high is to be compensated for obtaining excellent mechanical properties.

In order to obtain a more precise information in regard to the ions that are released by the respective glass ionomer particles and thus in regard to the ions that are available for the glass ionomer reaction with the respective acid-containing matrix, the ion release must be measured. Such measurements are carried out in water at constant temperatures and pH value (eg. 6.5 and 3.2) over a period of 24 hours. The samples taken at defined intervals are then quantitatively assayed by means of atomic adsorption spectroscopy or ICP analysis. Ion release values of 0.01 mg/l to 500 mg/l (values given as metal oxide) after 24 hours have been found to be very beneficial for the present invention. Release values of preferably 1 to 100 mg/l and particularly preferred of 10 to 50 mg/l have been found to be especially valuable. However, ionomer cements are also suitable whose ion release values are outside of the aforementioned broader range.

It should be noted that the ion release depends not only on the composition and temperature treatment of the ionomer particles but also greatly on their specific surface area; it is directly proportional thereto. Accordingly, greatly porous particles have generally higher release values and compact particles in principle have reduced release values.

After curing such systems, novel materials (composites, cement, compomers) are obtained that, while having a simpler composition and providing for a simpler as well as less expensive manufacture, have in comparison to know systems similar or significantly improved properties (x-ray adsorption, mechanical properties etc.).

For realizing optionally desired high x-ray opacity for reasons of biocompatibility primarily barium-poor or barium-free compositions are preferred. They may contain other heavy elements such as preferably Sr, Y, Sn and the lanthenoides or especially preferred Zr, Nb, or Ta.

Examples are provided in the following.

### EXAMPLE 1

### SiO₂/Al₂O₃ Particles; Weight Ratio 75/25

At room temperature with stirring, 30 ml water and glacial acetic acid were added to 7.9 g of aluminum secondary butylate. The resulting Al-containing solution was added dropwise to a dispersion that was obtained by dilution of 12.3 g of commercial SiO₂ sol (silica sol Ludox AS40, Du Pont company) with 75 g water and 2 ml glacial acetic acid. After spray drying at approximately 250° C., a white powder was obtained that according to REM images is comprised of approximately spherical particles. Measurements by means of x-ray fluorescence (XRF) confirmed a ratio of SiO₂/Al₂O₃ that is similar to the educt ratio. A temperature treatment by continuous heating in a muffle furnace up to 800° C. was carried out subsequently.

For determining the cement formation and thus the functionality of the prepared reactive particles, the particles were mixed with a commercially available poly carboxylic acid (polyacryic acid, MW 60,000) dissolved in water or a carboxylic add containing hybrid polymer (ORMOCER®) resin. Curing of the mixture by ionomer reaction occurred and was detected by means of FTIR spectroscopy of the COO—Al bond being formed with the aid of an asymmetric band at approximately 1,593 cm⁻¹.

### EXAMPLE 2

### SiO₂/CaO Particles; Weight Ratio 75/25

At room temperature with stirring, 20 ml water and 1 ml glacial acetic acid were added to 4.7 g of calcium acetate. The resulting Ca-containing solution was added dropwise to a dispersion that was obtained by dilution of 12.3 g of commercial SiO₂ sol (silica sol Ludox AS40, Grace Davison company) with 75 g water and 2 ml glacial acetic acid. After spray drying at approximately 250° C., a white powder was obtained that according to REM images is comprised of approximately spherical particles. XRF measurements confirmed a ratio of SiO₂/CaO that is similar to the educt ratio. A temperature treatment by continuous heating in a muffle furnace up to 800° C. was carried out subsequently. For determining the cement formation and thus the functionality of the prepared reactive particles, the particles were mixed with a commercially available poly carboxylic acid (polyacrylic acid, MW 60,000) dissolved in water or a carboxylic add containing hybrid polymer (ORMOCER®) resin. Curing of the mixture by ionomer reaction occurred and was detected by means of FTIR spectroscopy of the COO—Ca bond being formed with the aid of an asymmetric band at approximately 1,555 cm⁻¹.

### EXAMPLE 3

### Al₂O₃/CaO Particles; Weight Ratio 50/50

At room temperature with stirring, 30 ml water and glacial acetic acid were added to 7.7 g of aluminum secondary butylate. To this mixture, at room temperature with vigorous stirring, a solution of 4.5 g calcium acetate with 20 ml water and 1 ml glacial acetic acid was added with stirring and subsequently diluted with 50 g water. After spray drying at approximately 250° C., a white powder was obtained. XRF measurements confirmed a ratio of Al₂O₃/CaO that is similar to the educt ratio. A temperature treatment by continuous heating in a muffle furnace up to 800° C. was carried out subsequently.

For determining the cement formation and thus the functionality of the prepared reactive particles, the particles were mixed with a commercially available poly carboxylic acid (polyacrylic acid, MW 60,000) dissolved in water or a carboxylic acid containing hybrid polymer (ORMOCER®) resin. Curing of the mixture by ionomer reaction occurred and was detected by means of FTIR spectroscopy of the COO—Ca bond and the COO—Al bond being formed with the aid of asymmetric bands at approximately 1556 and 1,594 cm⁻¹.

### EXAMPLE 3

### Al₂O₃/SrO Particles; Weight Ratio 50150

At room temperature with stirring, 30 ml water and glacial acetic acid were added to 8.0 g of aluminum secondary butylate. To this mixture, at room temperature with vigorous stirring, a solution of 3.3 g strontium acetate with 20 ml water and 1 ml glacial acetic acid was added with stirring and subsequently diluted with 50 g water. After spray drying at approximately 250° C., a white powder was obtained. XRF measurements confirmed a ratio of Al₂O₃/SrO that is similar to the educt ratio. A temperature treatment by continuous heating in a muffle furnace up to 800° C. was carried out subsequently.

For determining the cement formation and thus the functionality of the prepared reactive particles, the particles were mixed with a commercially available poly carboxylic acid (polyacrylic acid, MW 60000) dissolved in water or a carboxylic acid containing hybrid polymer (ORMOCER®) resin. Curing of the mixture by ionomer reaction occurred and was detected by means of FTIR spectroscopy of the COO—Sr bond and the COO—Al bond being formed with the aid of asymmetric bands at approximately 1556 and 1,590 cm⁻¹.

### EXAMPLE 5

### SnO₂/CaO Particles, Weight Ratio 75/25

At room temperature with stirring 20 ml water and 1 ml glacial acetic acid were added to 4.7 g of calcium acetate. The resulting Ca-containing solution was added dropwise to a dispersion that was obtained by dilution of 33.3 g of commercial SnO₂ sol (15% aqueous dispersion, Alfa Aesar company) with 20 g water and 2 ml glacial acetic acid. After spray drying at approximately 240° C., a white powder was obtained that according to REM images is comprised of approximately spherical particles. XRF measurements confirmed a ratio of SiO₂/CaO that is similar to the educt ratio. A temperature treatment by continuous heating in a muffle furnace up to 500° C. was carried out subsequently. In between the temperature was maintained for a time period of 30 min at 300° C. The resulting particles have a diameter of 4.7 μm (volume distribution) measured by Fraunhofer diffraction. The specific surface area measured by N₂ sorption according to BET was 121 m²/g.

For determining the cement formation and thus the functionality of the prepared reactive particles, the particles were mixed with a commercially available poly carboxylic acid (polyacrylic acidc, MW 60,000) dissolved in water or a carboxylic acid containing hybrid polymer (ORMOCER®) resin. Curing of the mixture by ionomer reaction occurred and was detected by means of FTIR spectroscopy of the COO—Ca bond being formed with the aid of an asymmetric band at approximately 1,556 cm⁻¹.

### EXAMPLE 6

### SiO₂/CaO Particles; Weight Ratio 75/25; Containing F

At room temperature with stirring, 20 ml water and 1 ml glacial acetic acid were added to 4.7 g of calcium acetate. The resulting Ca-containing solution was added dropwise to a dispersion that was obtained by dilution of 12.3 g of commercial SiO₂ Sol (silica sol Ludox AS40, Grace Davison company) with 75 g water and 0.5 g hexafluoro silicic acid. After spray drying at approximately 250° C., a white powder was obtained that according to REM images is comprised of approximately spherical particles, XRF measurements confirmed a ratio of SiO₂/CaO that is similar to the educt ratio. A temperature treatment by continuous heating in a muffle furnace up to 800° C. was carried out subsequently.

For determining the cement formation and thus the functionality of the prepared reactive particles, the particles were mixed with a commercially available poly carboxylic acid (polyacrylic acid, MW 60,000) dissolved in ater or a carboxylic acid containing hybrid polymer (ORMOCER®) resin. Curing of the mixture by ionomer reaction occurred and was detected by means of FTIR spectroscopy of the COO—Ca bond being formed with the aid of an asymmetric band at approximately 1,554 cm⁻¹.

### EXAMPLE 7

### SnO₂/CaO Particles; Weight Ratio 75/25

At room temperature with stirring, 20 ml water and 1 ml glacial acetic acid were added to 4.7 g of calcium acetate. The resulting Ca-containing solution was added dropwise to a dispersion that was obtained by dilution of 33.3 g of commercial SnO₂ sol (15% aqueous dispersion, Alfa Aesar company) with 20 g water and 2 ml glacial acetic acid. After spray drying at approximately 240° C., a white powder was obtained that according to REM images is comprised of approximately spherical particles. XRF measurements confirmed a ratio of SiO₂/CaO that is similar to the educt ratio. A temperature treatment by continuous heating in a muffle furnace up to 800° C. was carried out subsequently and was supplemented by a holding time at 300° C. The resulting particles have a diameter of 4.5 μm (volume distribution) measured by Fraunhofer diffraction. The specific surface area measured by N₂ sorption according to BET was 76 m²/g.

For determining the cement formation and thus the functionality of the prepared reactive particles, the particles were mixed with a commercially available poly carboxylic acid (polyacrylic acid, MW 60,000) dissolved in water or a carboxylic acid containing hybrid polymer (ORMOCER®) resin. Curing of the mixture by ionomer reaction occurred and was detected by means of FTIR spectroscopy of the COO—Ca bond being formed with the aid of an asymmetric band at approximately 1,555 cm⁻¹.

What is claimed is:

1. Ionomer particles with an inner area and an outer area, wherein the outer area has an oxidic matrix with cations that are selected by observing the following conditions:
only two different types of cations must be present that belong to one of the following groups (a) to (d);
these two types of cations must be selected from two different ones of the following groups (a) to (d):
(a) ions of the elements of the second main group as well as bivalent ions of the transition elements and the lanthanides,
(b) ions of the elements of the third main group with the exception of boron as well as trivalent ions of the transition elements and the lanthanides,
(c) ions of the elements of the fourth main group with the exception of carbon as well as tetravalent ions of the transition elements and the lanthanides:
(d) ions of the elements of the fifth main group, selected from ions of antimony and bismuth as well as pentavalent ions of the transition elements;
with the proviso that particles of the composition ZrO₂—Y₂O₃, ZrO₂—Al₂O₃, BaTiO₃. Al₆Si₂O₁₃ are not contained.

2. Ionomer particles according to claim 1, with the with the proviso that particles of the composition 3 CaOxSiO₂ in as much as they are produced by application of temperatures above 1,000° C. and/or of particles of a combination of aluminum oxide or silicon dioxide with an oxide selected from the oxides of lanthanum, zirconium, and yttrium are not contained.

3. Ionomer particles according to claim 1, wherein the group (b) comprises the ions of the elements of the third main group with the exception of boron and of aluminum as well as trivalent ions of the transition elements and the lanthanides.

4. Ionomer particles according to claim 1, wherein the group (c) comprises the ions of the elements of the fourth main group with the exception of carbon and of silicon as well as tetravalent ions of the transition elements and the lanthanides.

5. Ionomer particles according to claim 1, wherein the oxidic matrix is homogeneous or substantially homogeneous.

6. Ionomer particles according to claim 1, with spherical or approximately spherical shape.

7. Ionomer particles according to claim 1, obtained by use of at least one of the two cations in dissolved form.

8. Ionomer particles according to claim 1, wherein the inner and outer areas are identical.

9. Ionomer particles according to claim 1, wherein the composition of the inner areas differs from that of the outer areas.

10. Ionomer particles according to claim 1, with a homogeneous distribution of the components in the respective areas.

11. Ionomer particles according to claim 1, with cluster-like areas of the components in the respective areas.

12. Ionomer particles according to claim 1, wherein the two cation types are selected from the groups (a) and (b) or from the groups (a) and (c) or from the groups (b) and (c) or from the groups (a) and (d), wherein the groups (a) and (b) are especially preferred.

13. Ionomer particles according to claim 9, wherein the inner area is comprised of silicon dioxide and tin dioxide.

14. Ionomer particle according to claim 12 in which the cations of the group (a) are calcium ions and/or strontium ions.

15. Ionomer particles according to claim 1, whose outer area is surface-modified by esterification with carboxylic acid or by silanization.

16. Method for producing ionomer particles according to claim 1, comprising the following steps:
(i) forming a dispersion, a suspension, a solution, an emulsion, a gel or a sol, by using
(1) two organic compounds containing one and the other of the two cation types, respectively, according to claim 1, or
(2) an organic compound with one of the cation types according to claim 1 and an oxide or an inorganic salt of the second cation
(3) two oxides of the two inorganic salts according to claim 1, in a suitable liquid medium
(ii) effecting an at least partial hydrolysis and condensation of the component(s) mentioned under (1) and optionally an at least substantially complete dissolution of the components under (2) or (3) so that a homogeneous or substantially homogeneous matrix results,
(iii) generating and optionally separating spherical or substantially spherical particles in or from the liquid medium, and
(iv) drying the spherical or substantially spherical particles.

17. Method according to claim 16, furthermore comprising the following steps: heating the dried particles to at least such a temperature at which optionally still present organic components of the particles are removed, but not above 1,000° C., preferably not above 650° C.

18. Method according to claim 16, wherein the particles are generated by an aerosol method, in particular spray drying.

19. Method according to claim 16, wherein the dispersion, suspension, the gel or sol additionally is realized by using oxidic particles whose diameter is below that of the ionomer particles to be produced, preferably in a range of 1-10%.

20. Method according to claim 16, additionally comprising the steps:
(v) generating a particle dispersion by careful optionally acid-catalyzed or base-catalyzed hydrolysis, of a metal alkoxide in an alcoholic solution, separation of the obtained particles from the suspension optionally drying of the particles, optionally heating of the particles in order to remove still present organic material, and using the particles for obtaining the dispersion, suspension, the gel or the sol according to (i) of claim 16.

21. Method according to claim 16, wherein the particles are formed by generating an emulsion, a dispersion, or a suspension whereupon they are removed from the surrounding solvent or the surrounding solvent is removed.

22. Kit for producing a cement, comprising ionomer particles according to claim 1 as well as a matrix curable with the aid of or in the presence of these ionomer particles.

23. Kit according to claim 22, wherein the matrix is a polymer matrix that comprises, or is, at least one carboxylic acid, preferably a polycarboxylic acid or a poly alkene acid.

24. Kit according to claim 22, wherein the polymer matrix is a carboxylic group-containing, homopolymeric for heteropolymeric matrix system, preferably one of at least one unsaturated mono-, di-, or higher polycarboxylic acid or its anhydride, to which are added optionally hydroxy carboxylic acids, in particular citric acid or tartaric acid.

25. Cement, produced by using a kit according to claim 22.

26. Cement, selected from the group of dental cements, bone cements and adhesives, comprising ionomer particles according to claim 1.

27. Cement according to claim 27, comprising as a further component a polymer matrix that comprises or is at least one carboxylic acid preferably a polycarboxylic acid or a poly alkene acid.

28. Cement according to claim 26, wherein the polymer matrix is a carboxyl group-containing homopolymeric or heteropolymeric matrix system, preferably one of at least one unsaturated mono- di-, or higher polycarboxylic acids or its anhydrides, having added thereto optionally hydroxy carboxylic acids, in particular citric acid or tartaric acid.

29. Cement according to claim 25, wherein the cement is a dental cement, bone cement, or adhesive for medical or non-medical purposes.
